(19) United States
(12) Patent Application Publication
(10) Pub. No.: US 2023/0302179 A1
(43) Pub. Date: Sep. 28, 2023

(54) MOVING-FRONT STERILIZATION MONITORING DEVICES

(71) Applicant: 3M INNOVATIVE PROPERTIES COMPANY, St. Paul, MN (US)

(72) Inventors: Joshua D. ERICKSON, Blaine, MN (US); Giuseppe M. BOMMARITO, Stillwater, MN (US); Ryan T. WOLDT, Minneapolis, MN (US); Timothy J. NIES, Stillwater, MN (US); Jeffrey M. FLYNN, Osceola, WI (US)

(21) Appl. No.: 18/040,882

(22) PCT Filed: Aug. 10, 2021
(86) PCT No.: PCT/IB2021/057369

§ 371 Date: Feb. 7, 2023

Related U.S. Application Data
(60) Provisional application No. 63/064,563, filed on Aug. 12, 2020.

(51) Int. Cl. A61L 2/20; A61L 2/24

(52) U.S. Cl. CPC A61L 2/208; A61L 2/24; A61L 2/206; A61L 2101/34

(57) ABSTRACT

The present disclosure relates to sterilization monitoring devices, as well as and methods and kits for evaluating sterilization processes using sterilization monitoring devices.

### BACKGROUND

Many objects require sterilization prior to use. Medical instruments, for example, are often sterilized in processes that involve contact with a sterilant, such as steam or other sterilizing liquids or gases, such as ethylene oxide or vaporized hydrogen peroxide. These processes achieve sterilization through the control of several physical and chemical parameters such as: temperature, pressure and sterilant concentration and physical state. Many objects require different sterilization processes due to, for example, object shape, types of contaminants, and/or the inclusion of heat-sensitive materials.

It is critical to have means for ensuring complete sterilization. Current strategies employ devices having either a chemical indicator or a biological indicator that allow a user to determine if the device has been sufficiently exposed to sterilant. For example, if the device has been sufficiently exposed to a sterilant, the chemical indicator may change color indicating the sterilization was successful. These devices are generally designed to mimic the challenges relative to a standardized load, i.e., a typical load comprising a certain number and/or type(s) of object. However, such devices are unable to accurately assess loads that deviate from the standard, e.g., loads with a greater number of tools and/or loads having particularly challenging tools, such as those with cavities and lumens that are difficult for a sterilant to access. In other words, current devices are not universal.

Further, it is critical to have means for monitoring sterilization processes. Current sterilization devices are unable to show inconsistencies between sterilization cycles since they only provide pass/fail information. Inconsistencies may indicate that a machine is in decline and/or that the machine is not providing adequate sterilization uniformly throughout the load. For example, ineffective chamber evacuation, insufficient sterilant concentration, machine leaks, or otherwise inadequate temperature or pressure may compromise the sterilization process.

### SUMMARY

Disclosed is a sterilization monitoring device that can function over a range of sterilization conditions because it can provide information as to the degree of sterilization. This ability to show a sterilization gradient can provide information as to machine consistency and performance, and also a device that allows for more than one sterilization modality.

In one embodiment, a sterilization monitoring device is described. The sterilization monitoring device may include a challenge network and a shell encasing the challenge network. The challenge network may have a flow channel and a plurality of indicator compartments. The flow channel may include an entry port for receiving a sterilant. Each indicator compartment may include one or more indicator and be in fluid communication with the flow channel. The shell may allow sterilant access to the plurality of indicator compartments only via the flow channel.

In one embodiment, a sterilization monitoring device is described. The sterilization monitoring device may include a plurality of challenge networks, at least one medium compartment, and a shell encasing the challenge networks. Each challenge network may include a flow channel having an entry port for receiving a sterilant, and one or more indicator compartment comprising one or more indicator, wherein at least one indicator is a biological indicator. The medium compartment may include a nutrient medium. The nutrient medium may be in fluid communication with one or more indicator compartment.

In one embodiment, a method for evaluating a sterilization process is described. The method may include providing a sterilization monitoring device; exposing the sterilization monitoring device to conditions set forth in a sterilization protocol. The sterilization protocol may include allowing a sterilant to contact the sterilization monitoring device for a period; inspecting one or more indicators for a change in one or more of color, pH, and fluorescence; comparing the change in one or more of color, pH, and fluorescence with a sterilization threshold value; and determine whether or not the change in one or more of color, pH, and fluorescence indicates a satisfactory sterilization in view of the sterilization threshold value.

In one embodiment, a kit is described. The kit may include a sterilization monitoring device described herein and instructions directing a user to carry out a method for evaluating a sterilization process described herein.

### BRIEF DESCRIPTION OF DRAWINGS

FIG. 1A illustrates one embodiment of the present disclosure having all indicator compartments situated along a flow channel of a single challenge network.

FIG. 1B is a perspective view from point B of the embodiment in FIG. 1A.

FIG. 1C is a perspective view from point C of the embodiment in FIG. 1A.

FIG. 2A illustrates one embodiment of the present disclosure having indicator compartments situated along sub-flow channels of a single challenge network.

FIG. 2B is a perspective view from point B of the embodiment in FIG. 2A.

FIG. 2C is a perspective view from point C of the embodiment in FIG. 2A.

FIG. 3A illustrates one embodiment of the present disclosure having indicator compartments and medium compartments situated along a flow channel of a single challenge network.

FIG. 3B is a perspective view from point B of the embodiment in FIG. 3A.

FIG. 3C is a perspective view from point C of the embodiment in FIG. 3A.

FIG. 4A illustrates one embodiment of the present disclosure having a plurality of challenge networks.

FIG. 4B is a perspective view from point B of the embodiment in FIG. 4A.

FIG. 5A illustrates one embodiment of the present disclosure having a plurality of challenge networks, each having a different configuration.

FIG. 5B is a perspective view from point B of the embodiment in FIG. 5A.

FIG. 6A illustrates an embodiment of the present disclosure having a plurality of challenge networks having medium compartments.

FIG. 6B is a perspective view from point B of the embodiment in FIG. 6A.

FIG. 7A illustrates an embodiment of the present disclosure having a plurality of challenge networks having medium compartments.

FIG. 7B is a perspective view from point B of the embodiment in FIG. 7A.

FIG. 8 illustrates one embodiment of the present disclosure having indicator compartments situated along a flow channel and a means to introduce a medium.

FIG. 9 illustrates one embodiment of the present disclosure having indicator compartments situated along sub-flow channels.

FIG. 10 is a graph depicting fluorescence data of one embodiment of the present disclosure after 3 min. exposure to steam.

FIG. 11 is a graph depicting fluorescence data of one embodiment of the present disclosure after 6 min. exposure to steam.

FIG. 12 is a flow diagram detailing method for evaluating a sterilization protocol of the present disclosure.

FIG. 13 is a diagram of a kit of the present disclosure.

### DETAILED DESCRIPTION

Sterilization monitoring devices having a moving-front and methods employing moving-front sterilization monitoring devices for evaluating a sterilization process are described. The devices of the present disclosure have several advantages over conventional devices. For example, most known devices include one indicator along a sterilant flow channel. Such devices are only capable of telling a user whether a sterilization process passed or failed according to the tolerance of that particular device. Known devices that offer an indicator at more than one sterilant flow channel (of different lengths) simply allow for a user to determine if sufficient time was allotted for a given process.

Conversely, moving-front devices are equipped with a plurality of indicators along a single a flow channel, which in many ways allows for a more universal application. The present devices may include a variety of indicators, different types of indicators, and indicators at different concentrations within a single device. Thus, these devices are equipped for use in any one of many sterilization protocols. Furthermore, a user may even use these device for subsequent sterilizations under different conditions.

In addition, moving-front devices lend to an increase in flow resistance the further a sterilant travels along the flow channel. In some cases, the increase in flow resistance may mimic non-standard sterilization loads, such as loads having objects with hard-to-access shapes, or particularly heavy loads having more objects than usual. In other words, indicators situated further along the flow channel may be relied upon to identify sterilization processes suitable for larger and/or diverse loads.

Lastly, the sterilization monitoring devices described herein may further monitor the consistency between cycles of a sterilization machine. Fluctuations in the number of indicators influenced by a sterilant, i.e., the reaches of the moving-front, may indicate to a user that a sterilization machine may require maintenance. In some instances, early maintenance may prevent a machine from being pulled from of operation for repair—an event that could cripple a hospital.

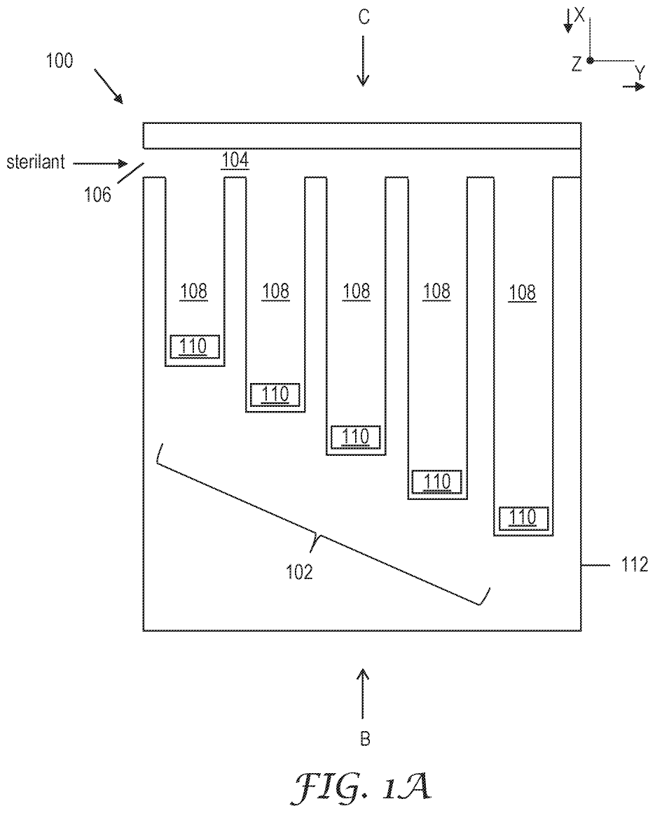

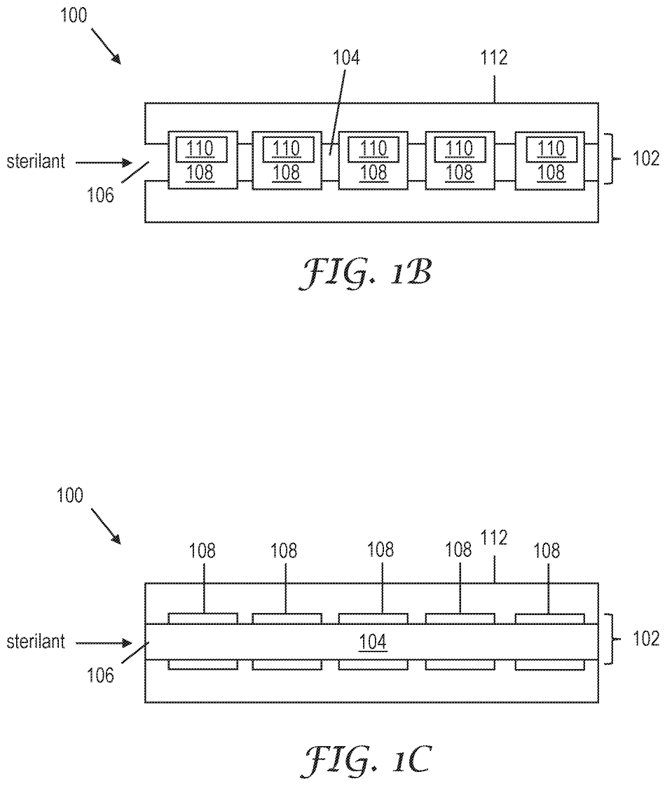

FIG. 1A illustrates a sterilization monitoring device 100 having a challenge network 102 including a flow channel 104 having an entry port 106 for receiving a sterilant, and a plurality of indicator compartments 108. Entry port 106 is the only way in which sterilant may enter the device. As shown, each indicator compartment 108 includes one or more indicator 110. Sterilization monitoring device 100 further includes a shell 112 encasing the challenge network. The direction in which sterilization monitoring device 100 is oriented is with respect to the direction of gravity being in the X direction, as indicated by the illustrated coordinate diagram. As shown, point C is the top of the device and point B is the bottom of the device.

FIG. 1B is a perspective view of sterilization monitoring device 100, as viewed from point B (bottom).

FIG. 1C is a perspective view of sterilization monitoring device 100, as viewed from point C (top).

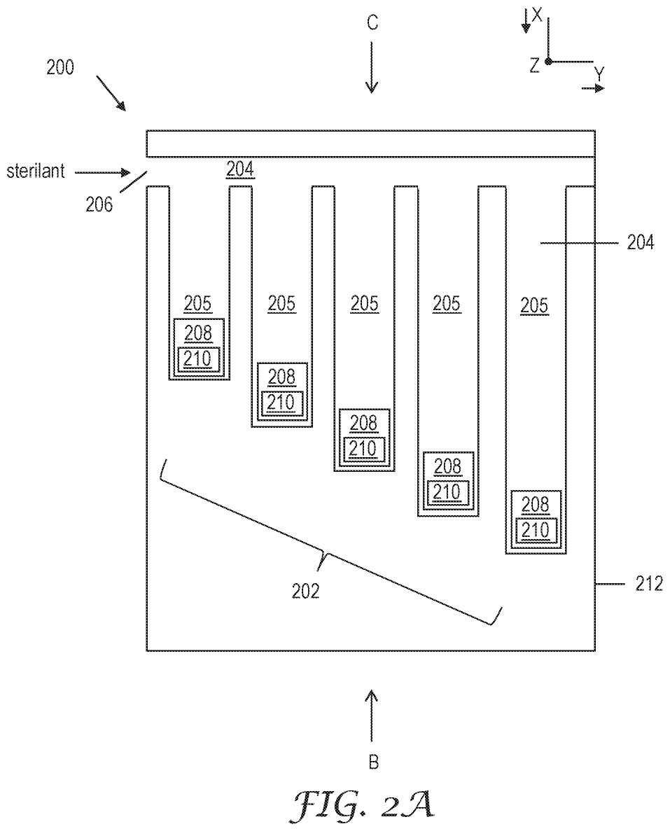

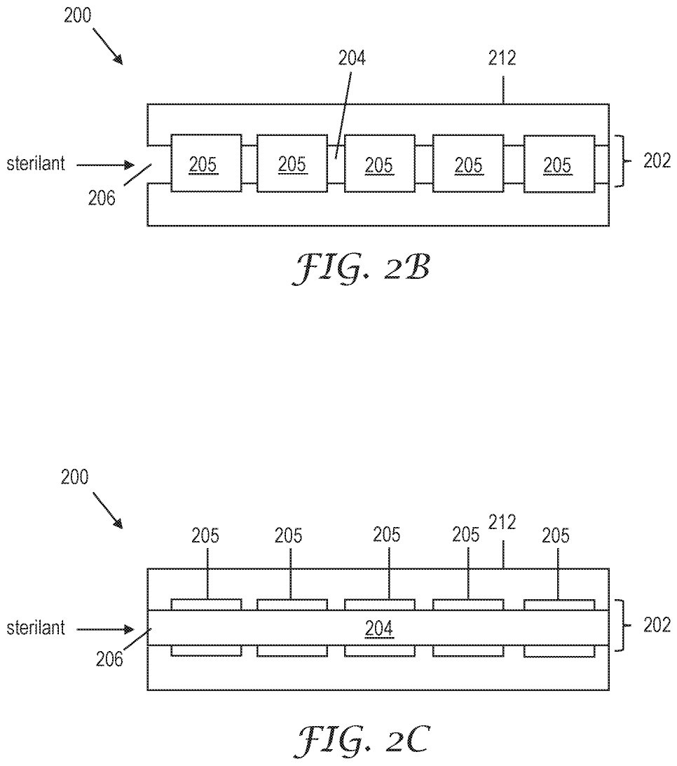

FIG. 2A illustrates a sterilization monitoring device 200 having a challenge network 102 including a flow channel 204 having an entry port 206 for receiving a sterilant, and a plurality of sub-flow channels 205. As shown, each of the sub-flow channels include an indicator compartment 208 housing one or more indicator 210. Sterilization monitoring device 200 further includes a shell 212 encasing the challenge network. The direction in which sterilization monitoring device 100 is oriented is with respect to the direction of gravity being in the Z direction, as indicated by the illustrated coordinate diagram. As shown, point C is one side of the device and point B is an opposite side of the device.

FIG. 2B is a perspective view of sterilization monitoring device 200 from point B.

FIG. 2C is a perspective view of sterilization monitoring device 200 from point C.

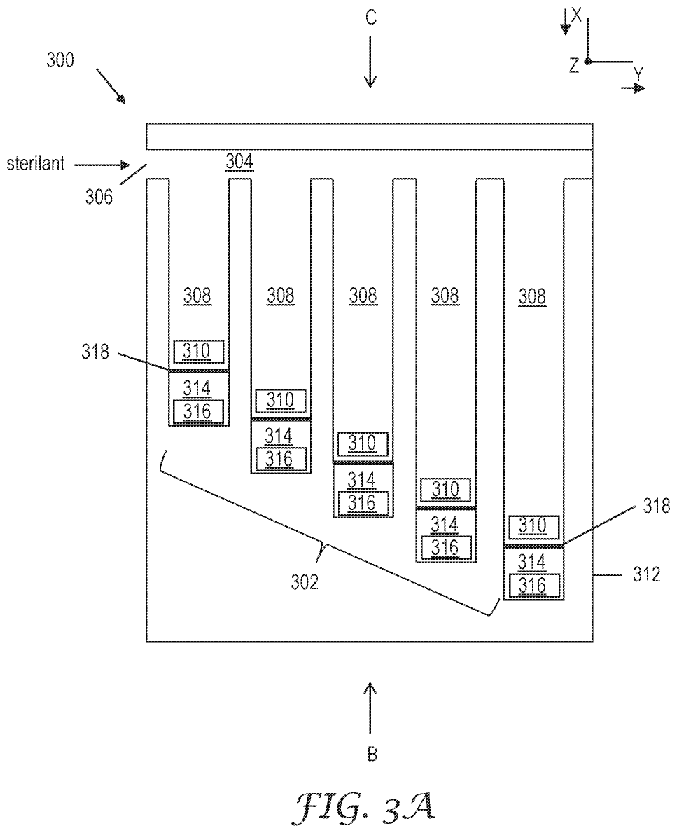

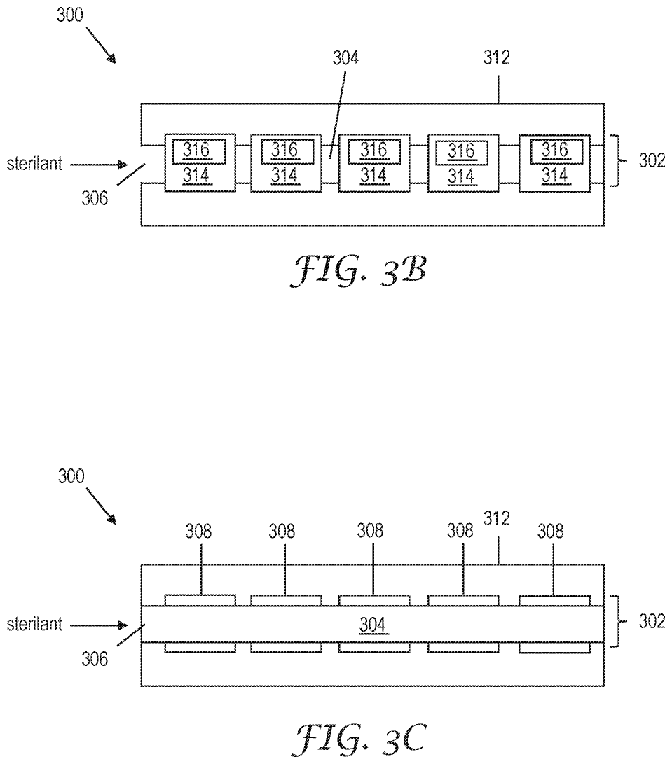

FIG. 3A illustrates a sterilization monitoring device 300 having a challenge network 302 including a flow channel 304 having an entry port 306 for receiving a sterilant, and a plurality of indicator compartments 308. As shown, each indicator compartment 308 includes one or more indicator 310. A shell 312 encasing the challenge network. Sterilization monitoring device 300 further includes medium compartments 314 having one or more nutrient mediums 316. Medium compartments 314 are separated from indicator compartments 308 by a barrier 318. The direction in which sterilization monitoring device 100 is oriented is with respect to the direction of gravity being in the X direction, as indicated by the illustrated coordinate diagram. As shown, point C is the top of the device and point B is the bottom of the device.

FIG. 3B is a perspective view of sterilization monitoring device 300 from point B.

FIG. 3C is a perspective view of sterilization monitoring device 300 from point C.

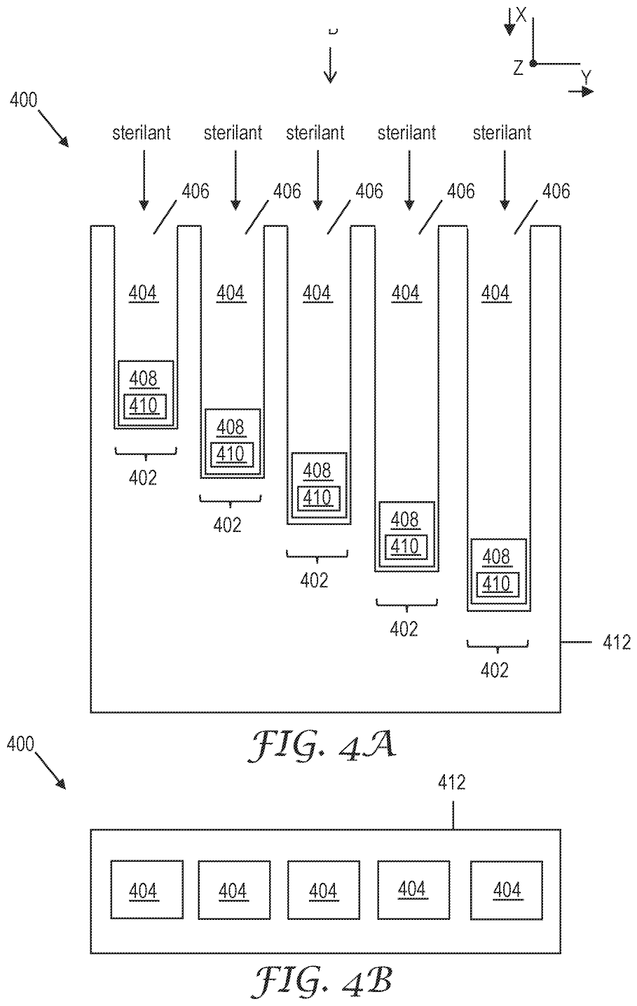

FIG. 4A illustrates a sterilization monitoring device 400 having a plurality of challenge networks 402. Each challenge network 402 includes a flow channel 404 having an entry port 406 for receiving a sterilant, and one or more indicator compartments 408 including one or more indicators 410. Sterilization monitoring device 400 further includes a shell 412 at least partly encasing challenge networks 402. The device may be oriented such that gravity is in the X, Y, or Z direction.

FIG. 4B is a perspective view of sterilization monitoring device 400 from point B.

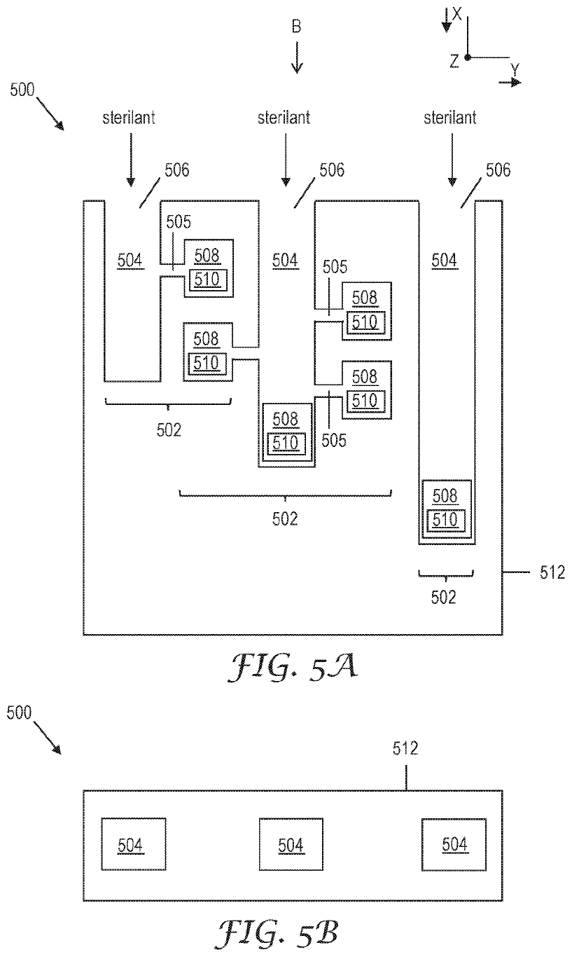

FIG. 5A illustrates a sterilization monitoring device 500 having a plurality of challenge networks 502. Each challenge network 502 includes a flow channel 504 having an entry port 506 for receiving a sterilant, and one or more indicator compartments 508 including one or more indicators 510. Indicator compartments 508 are located along one or more of flow channel 504 and sub-flow channel(s) 505. Sterilization monitoring device 500 further includes a shell 512 at least partly encasing challenge networks 502. The device may be oriented such that gravity is in the X, Y, or Z direction.

FIG. 5B is a perspective view of sterilization monitoring device 500 from point B.

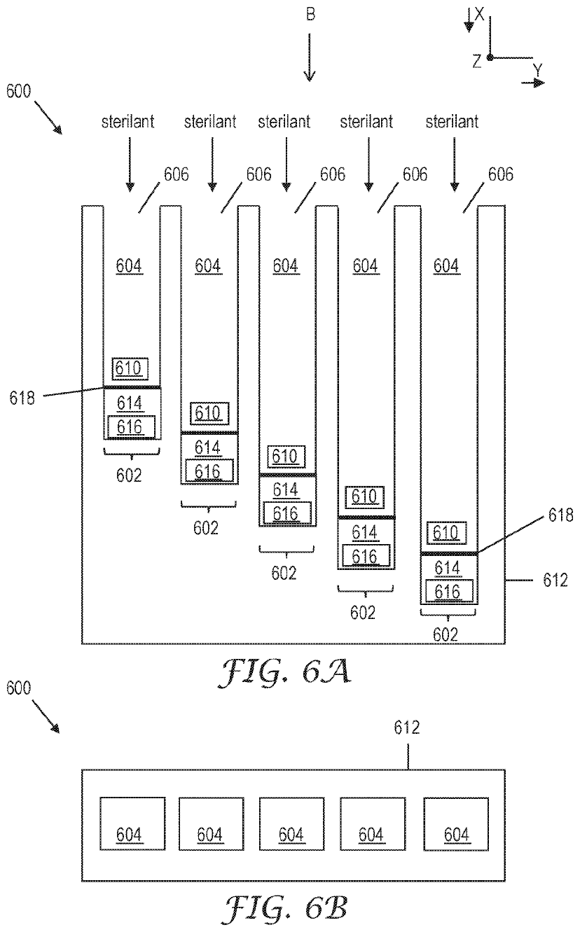

FIG. 6A illustrates a sterilization monitoring device 600 having a plurality of challenge networks 602. Each challenge network 602 includes a flow channel 604 having an entry port 606 for receiving a sterilant, and one or more indicator compartments 608 including one or more indicators 610. Sterilization monitoring device 600 further includes medium compartments 614 having one or more nutrient mediums 616. Medium compartments 614 are separated from indicator compartments 608 by a barrier 618. A shell 612 at least partly encases challenge networks 602. The device may be oriented such that gravity is in the X, Y, or Z direction.

FIG. 6B is a perspective view of sterilization monitoring device 600 from point B.

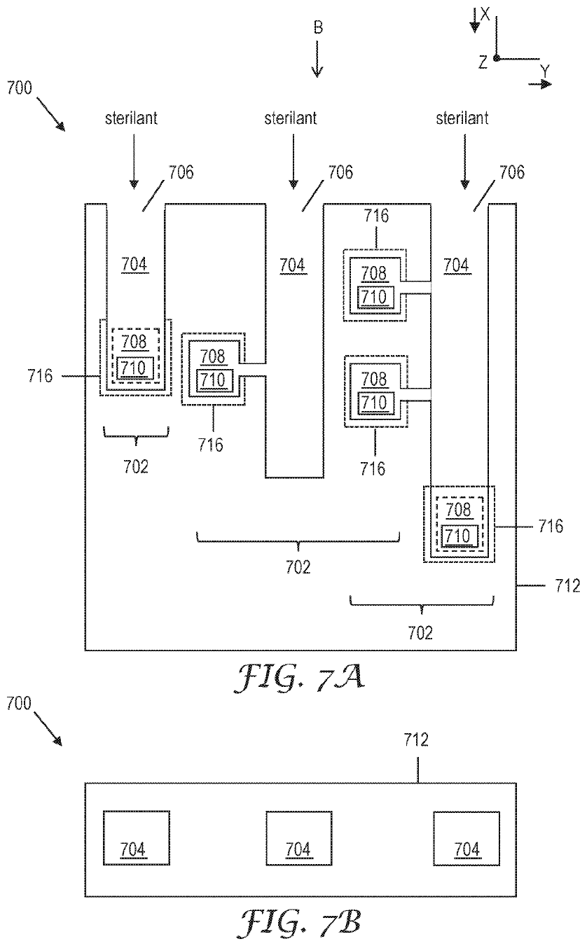

FIG. 7A illustrates a sterilization monitoring device 700 having a plurality of challenge networks 702. Each challenge network 702 includes a flow channel 704 having an entry port 706 for receiving a sterilant, and one or more indicator compartments 708 including one or more indicators 710. As shown, device 700 includes medium compartments 716 below indicator compartments 708. Sterilization monitoring device 700 further includes a shell 712 at least partly encasing challenge networks 702. The device may be oriented such that gravity is in the X, Y, or Z direction.

FIG. 7B is a perspective view of sterilization monitoring device 700 from point B.

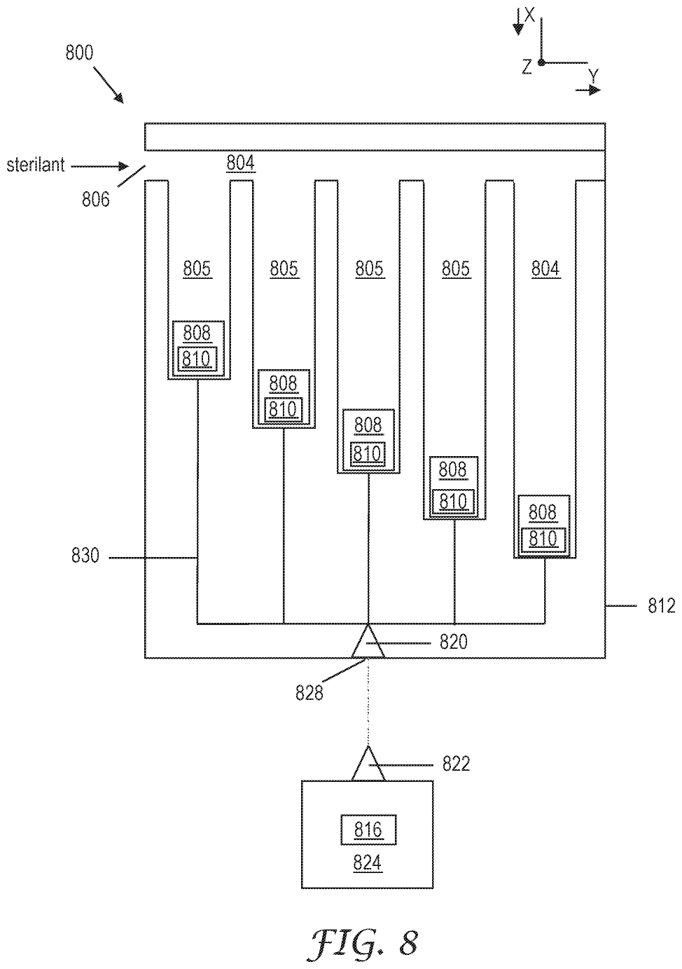

FIG. 8 illustrates a sterilization monitoring device 800 having a challenge network 802 including a flow channel 804 having an entry port 806 for receiving a sterilant. Flow channel 804 has a plurality of sub-flow channels 805. As shown, each sub-flow channel includes an indicator compartments 808 having one or more indicator 810. Sterilization monitoring device 800 further includes a shell 812 encasing challenge network 802. Shell 812 includes a medium port 820 configured to accept a medium output 822 of a medium packet 824 comprising a medium 816. Insertion of medium output 822 through a port barrier 828 into medium port 820 allows for medium 816 to be expelled into a medium path 830 that extends into indicator compartments 808 such that medium 816 interacts with the one or more indicators 810. The device may be oriented such that gravity is in the X, Y, or Z direction.

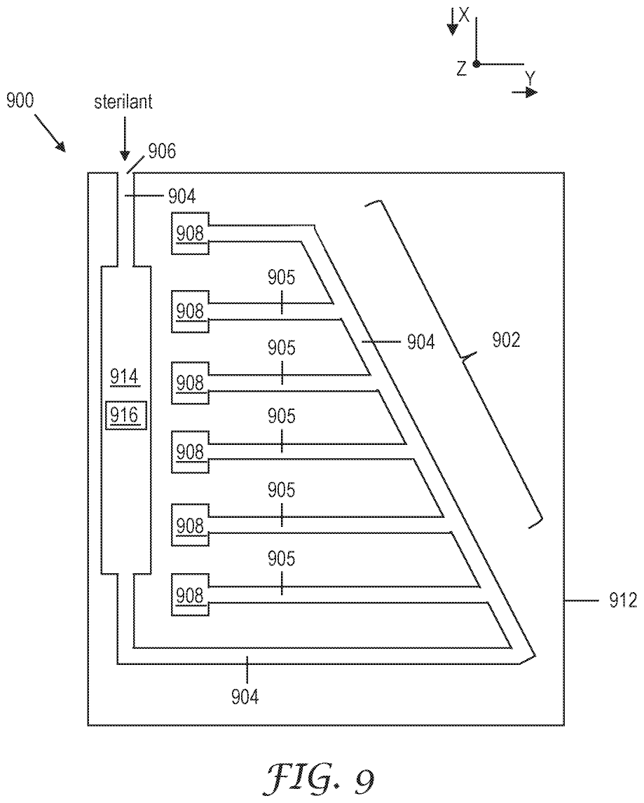

FIG. 9 illustrates a sterilization monitoring device 900 having a challenge network 902 including a flow channel 904 having an entry port 906 for receiving a sterilant. Flow channel 904 is shown to have a plurality of sub-flow channels 905. Each sub-flow channel includes an indicator compartment 908 having one or more indicator (not shown). In this embodiment, sterilization monitoring device 900 further includes a medium compartments 914 having one or more nutrient mediums 916. A shell 912 encases challenge network 902. The device may be oriented such that gravity is in the X, Y, or Z direction.

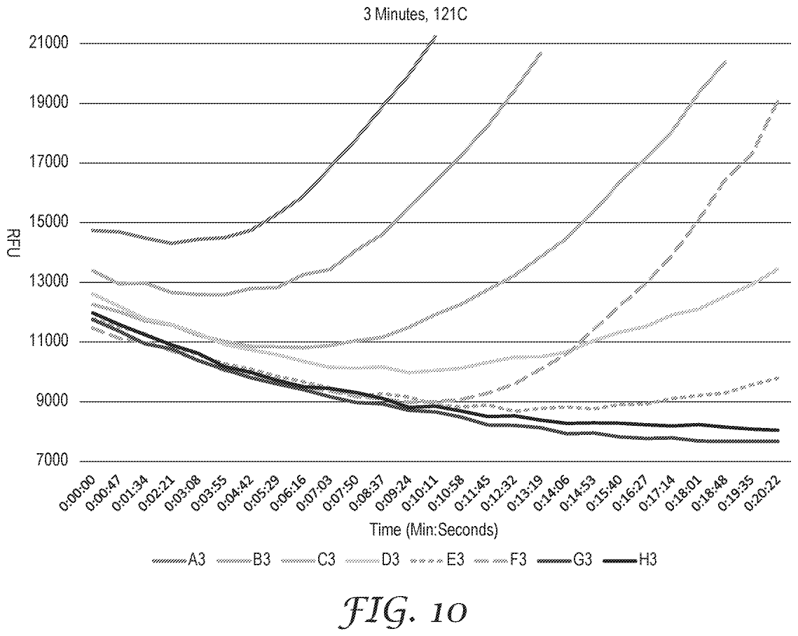

FIG. 10 is a graph depicting fluorescence data of one embodiment of the present disclosure resembling FIG. 1A-C after 3 min. exposure to steam.

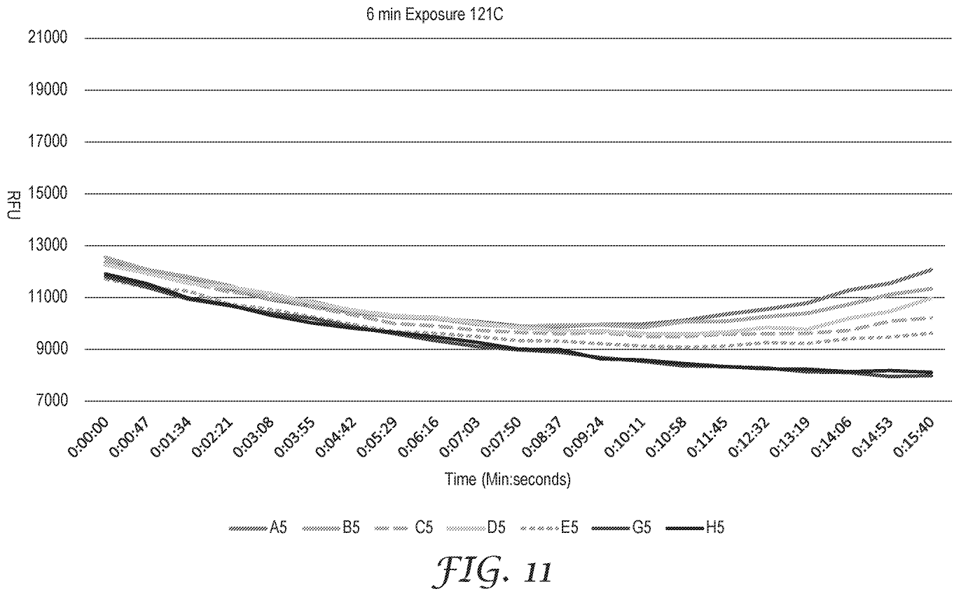

FIG. 11 is a graph depicting fluorescence data of one embodiment of the present disclosure resembling FIG. 1A-C after 6 min. exposure to steam.

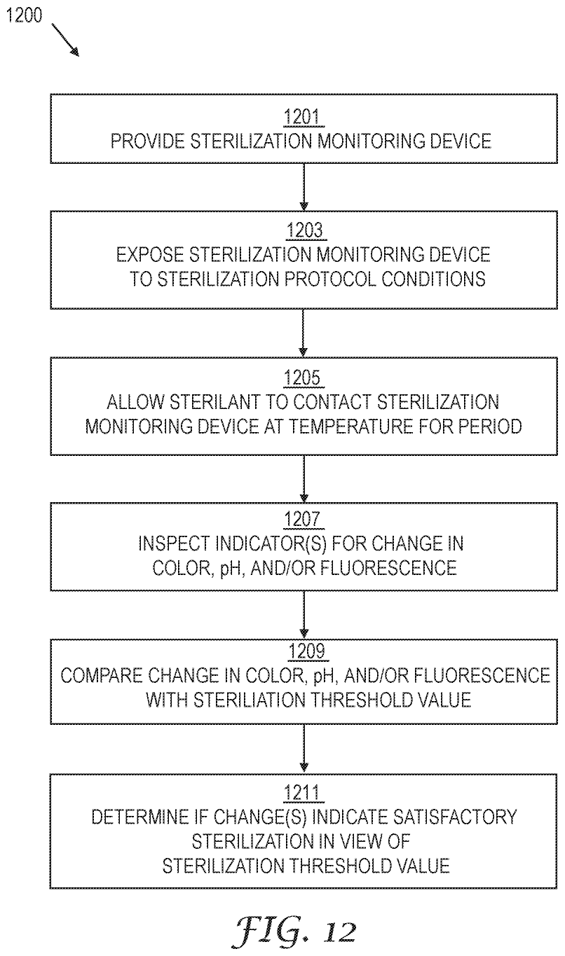

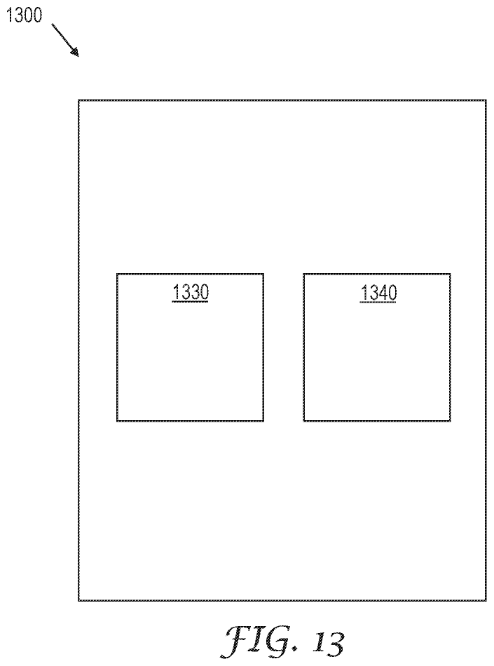

FIG. 12 is a flow chart illustrating the method 1200 for evaluating a sterilization protocol. The method may include 1201 providing a sterilization monitoring device described herein, 1203 exposing the sterilization monitoring device to sterilization protocol conditions, 1205 allowing sterilant to contact the sterilization monitoring device at a temperature and pressure for a time period, 1207 inspecting indicator(s) for a change in indicator state, (e.g., color, pH, fluorescence, or the like, or a combination thereof), 1209 comparing the change in indicator state with a sterilization threshold value, and 1211 determining if the change(s) indicate satisfactory sterilization in view of the sterilization threshold value.

FIG. 13 is a diagram of a kit 1300 having a sterilization monitoring device 1330 described herein and a set of instructions 1340 directing a user to carry out a method for evaluating a sterilization protocol.

The sterilization monitoring devices disclosed herein are placed within a sterilant chamber and subjected to an environment of sterilant under a set of conditions characterized at least by temperature, pressure, and time. The devices are often accompanied by an instrument load(s) that require sterilization. Sterilant enters the entry port(s) of the sterilization monitoring device and travels along the challenge network toward the indicator compartment(s). As the sterilant progresses through the flow channel, the sterilant experiences an increase in resistance (i.e., flow resistance). In other words, it is more difficult for the sterilant to travel deeper into the device. Thus, indicator compartments located further along the challenge network are more challenging for the sterilant to reach. This device design allows for universal monitoring of various sterilization protocols within a single device. For example, a sterilization protocol “A” may have a sterilization threshold value (for a given device and/or load class) of “n” number of indicators that are required to have undergone a change in indicator state in order to indicate a satisfactory sterilization (i.e., the load is appropriately sterilized for use), whereas a sterilization protocol “B” may have a sterilization threshold value of “n+3” number of indicators that are required to have undergone a change in indicator state in order to indicate a satisfactory sterilization, or the like.

Upon completion of a sterilization process, biological indicators may be supplied with nutrients to promote the growth of surviving organisms. Such nutrient medium(s) may be provided from a chamber internal or external to the device as described herein.

It should be understood that the illustrated embodiments within the Figures may not be drawn to scale or limited in design or number of any components shown. Any components described herein may be combined or substituted based on the desired properties of the device.

As used herein, “about” is used in conjunction with numerical values to include normal variations in measurements as expected by persons skilled in the art, and understood to have the same meaning “approximately.” The range intended to be encompassed by the term “about” is ±5% of the stated value.

As used herein, “biological indicator” refers to a biological composition that changes from one measurable state to another measurable state upon exposure to sufficient sterilant. A biological composition includes microorganism(s), such as bacterial spores or endospores, that may change in biological activities upon exposure to sufficient sterilant.

As used herein, “cavity volume” refers to the total volume with respect to the sum of volumes for each indicator compartment within a given challenge network. Increasing the volume of one or more indicator compartments would provide an increase in cavity volume, thereby increasing the flow resistance of a chosen sterilant.

As used herein, “chemical indicator” refers to a non-biological composition that changes from one measurable state to another measurable state upon exposure to sufficient sterilant. A non-biological composition, for example, includes compounds that change oxidation state, change color, change fluorescence, change pH, or the like, when exposed to sufficient sterilant.

As used herein, “flow resistance” refers to a resistance exerted on a sterilant traveling along a challenge network of a sterilization monitoring device described herein. For example, flow resistance may be increased by designing a sterilization monitoring device having a higher cavity volume and/or a lower hydraulic radius, e.g., larger indicator compartments, more indicator compartments, smaller flow channel cross-sectional areas, or the like. For example, flow resistance may be increased by designing a sterilization monitoring device having a challenge network with a greater number of sub-flow channels. For example, flow resistance may be increased by designing a sterilization monitoring device a challenge network allowing for sterilant flow in more than one plane of flow, e.g., flow of sterilant perpendicular or parallel to the direction of gravity, or the like. Another example of modifying flow resistance may include incorporating a sterilant-absorbent material within one or more of the flow channel and sub-flow channel. The flow resistance is dependent upon the type of sterilant used.

As used herein, “flow channel” refers to the longest path within a challenge network that a sterilant may travel. All paths extending from the longest path are considered “sub-flow channels” herein.

As used herein, “hydraulic radius” refers to the cross-sectional area of a flow and/or sub-flow channel with respect to the wetted perimeter of the conduit. Calculating the hydraulic radius (RH) is known in in the art: R_{H}=A/P, where A is the cross-sectional area and P is the wetted perimeter. “Wetted perimeter” refers to the perimeter that is contacted by the sterilant. Common calculations can be found at:

https://www.brighthubengineering.com/hydraulics-civil-engineering/67126-calculation-of-hydraulic-radius-for-uniform-open-channel-flow/. The hydraulic radius is dependent upon sterilant choice. Diffusion of a chosen sterilant into and through a flow channel and cavity volume is dependent on the sterilant temperature, concentration and time of exposure. The physical model describing sterilant diffusion and reaction with an indicator is characterized by the following linear partial differential equation:

$$\frac{\partial c}{\partial t} = D \frac{{\partial }^{2} c}{\partial {x}^{2}}$$

where D is the diffusion coefficient of the sterilant, c is the sterilant concentration, t is time and x is the distance along the channel of the integrator. The partial differential equation can be solved using an initial condition and two boundary conditions. The resulting solution represented by the following:

$$c = \overline{c} + \frac{Δ c}{2} erf ( \frac{x}{\sqrt{4 D t}} )$$

is an error function that depends on all the critical parameters associated with the sterilization cycle. Changing the sterilant concentration will change the initial condition and the resulting concentration profiles in the flow channel, whereas changing the sterilant type and temperature will change the diffusion constant. The diffusion length is represented by the following:

l_{D}=\sqrt{4Dt}

and is proportional to:

$${l}_{D} ∝ \frac{{V}_{C a v i t y}}{{r}_{h}^{2}}$$

where V_{cavity} is the cavity volume and rₕ is the hydraulic radius of the flow channel.

As used herein, “indicator” refers to a chemical or biological composition that is changed from one detectable state to another detectable state upon exposure to a sufficient amount of sterilant.

As used herein, “load class” refers to an instrument classification. For example, a sterilization load comprising instruments having access-challenged cavities may be of a different load class than, for example, a load comprising instruments without access-challenged cavities. A load class can be determined according to, for example, load weight, number of instruments, instruments of different material and shape, degree of contamination, or the like. A load class can be standardized (e.g., X number of instruments A, Y number of instruments B, etc.).

As used herein, “moving-front” refers to the length along a flow channel in which a sterilant flows. A moving-front also refers to the number of indicators influenced by a sterilant along a flow channel. As used herein, ‘influenced” refers to indicators that have been changed from one detectable state to another detectable state, e.g., spores initially present vs. spores reduced/killed.

As used herein, “satisfactory” refers to a pass (satisfactory) or fail (unsatisfactory) determination. A satisfactory sterilization means that the sterilization process successfully sterilized a particular load in view of the number of indicators having undergone a change in indicator state at or above the sterilization threshold value (for the particular load class).

As used herein, “sterilization threshold value” is a value referring to a number of indicators within a given device that are required to have undergone a change in indicator state based on a given sterilization protocol, i.e., sterilant type, temperature, pressure, time, load class, and the like. A sterilization threshold value can be determined for a standardized load class, for example, by subjecting a load to a sterilization protocol in the presence of a known sterilization monitoring device and a sterilization monitoring device described herein. Additionally, or alternatively, loads may independently be subjected to varying protocols and later tested for surviving organisms.

As used herein, “substantially impermeable” refers to the relative inability of a sterilant to penetrate the article to which it described. An article that is substantially impermeable to a sterilant allows less than about 1%, e.g., less than 0.1, 0.2, 0.3, 0.4, 0.5, 0.6 0.7, 0.8, 0.9, or 1, of sterilant flowing past the flow channel entry. An article that is substantially impermeable to sterilant, such as a shell of the present disclosure, does not allow sterilant to interact with an indicator within the shell above the specified percentage. One may test for permeability by blocking the flow channel entrance prior to exposing the device to a sterilant conditions. If the indicators are changed, then the shell is not considered substantially impermeable to sterilant for the purposes of the present disclosure.

### Sterilization Monitoring Device

In various embodiments, a sterilization monitoring device is described. The sterilization monitoring device may include a challenge network and a shell encasing the challenge network. The challenge network may have a flow channel and a plurality of indicator compartments. The flow channel may include an entry port for receiving a sterilant. Each indicator compartment may include one or more indicator and be in fluid communication with the flow channel. The shell may allow sterilant access to the plurality of indicator compartments only via the flow channel. All features may be selected, or selected in combination, from the features described in more detail below.

In various embodiments, a sterilization monitoring device is described. The sterilization monitoring device may include a plurality of challenge networks, at least one medium compartment, and a shell encasing the challenge networks. Each challenge network may include a flow channel having an entry port for receiving a sterilant, and one or more indicator compartment comprising one or more indicator, wherein at least one indicator is a biological indicator. The medium compartment may include a nutrient medium. The nutrient medium may be in fluid communication with one or more indicator compartment. All features may be selected, or selected in combination, from the features described in more detail below.

In some embodiments, the sterilization monitoring device is configured to indicate the degree of sterilization for a given sterilization protocol. In other words, the sterilization monitoring device provides information beyond a pass/fail tolerance. Often times pass/fail mechanisms are subject to being misinterpreted by an evaluator and may not provide universal assessment, e.g., for particularly heavy sterilization loads, or loads including instruments having access-challenged cavities. The sterilization monitoring devices of the present disclosure allow for evaluation beyond pass/fail. The number of indicator compartments within a single challenge network having indicators influenced by the sterilant at or above a sterilization threshold value raises the confidence interval. For example, X number of indicators at an appropriate sterilization threshold may be required for a passing a given sterilization load. In some examples, X+1, X+2, X+3 indicators, and so forth, at an appropriate sterilization threshold would provide greater confidence in the sterilization process.

In some embodiments, the sterilization monitoring device is configured to detect a change in the workability of a sterilizer machine. For example, a user may be able to observe a decline in sterilization capacity of a machine, whether it be due to problems with chamber evacuation, leaks, sterilant pressure, or the like. For example, a sterilizer machine may operate consistently at a level to provide Y number of indicators at an appropriate sterilization threshold for a standard load. An observed drop in the number of indicators at an appropriate sterilization threshold, e.g., Y−1, Y−2, or the like, may notify a user that the machine may need minor repair or adjustment that could prevent further damage. In many instances, a compromised sterilizer in a hospital setting may put people in danger that are depending on doctors and surgeons having sterile tools available.

In some embodiments, the sterilization monitoring device may further include more than one challenge network. Each challenge network may independently be in the same or different spatial configuration. Each challenge network may independently have the same or different number of indicator compartments. Each challenge network may independently have the same or different types of indicators.

In some embodiments, the sterilization monitoring device may further include one or more sub-flow channel extending from the flow channel. The one or more sub-flow channel may be any sub-flow channel described herein.

In some embodiments, the sterilization monitoring device may further include one or more medium compartment. A medium compartment may house a medium selected from a nutrient medium, a fluorogenic medium, a pH indicator medium, and a combination thereof The medium compartment(s) may independently include any medium described herein.

In some embodiments, the sterilization monitoring device may further include a medium port for receiving medium via a separate medium source. The medium port may be in fluid communication with one or more indicator compartment via a medium pathway. In such configuration, a user may inject medium into the sterilization monitoring device such that the medium travels through the medium pathway and enters the indicator compartment(s).

In some embodiments, the sterilization monitoring device may have a cavity volume of about 0.01 mL to 10 mL. For example, the cavity volume may be a value in mL selected from 0.01, 0.02, 0.03, 0.04, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 and 10.0, or a value in a range between any of the preceding values, for example, between about 3.0 and about 7.0, between about 0.05 and about 5.5, or the like.

In some embodiments, the sterilization monitoring device may include a challenge network configured to accept any sterilant described herein under any conditions described herein. The sterilant may be in a liquid or gaseous state.

In some embodiments, the sterilization monitoring device may include a medium port described herein for receiving a medium described herein. In some embodiments, the sterilization monitoring device may include a medium path described herein. One or more indicator compartment may be in fluid communication with the medium port via the medium path.

In some embodiments, the sterilization monitoring device may further include a sterilant-permeable package at least partly surrounding the shell. The sterilant-permeable package may increase a sterilant flow resistance.

All features of the sterilization monitoring devices are described in further detail below.

### Flow Channel

In some embodiments, the flow channel may have a length of about 10 mm to about 800 mm. A flow channel length is a measure of the total distance a sterilant may travel. For example, the flow channel may have a length in mm of about 10, 20, 30, 40, 50, 60, 70, 80, 90 100, 150, 200, 250, 300, 350, 500, 450, 500, 550, 600, 650, 700, 750, or 800, or a value in a range between any of the preceding values, for example, between about 400 and about 600, between about 100 and about 300, or the like.

In some embodiments, the flow channel may have a maximum cross-sectional width of about 0.125 mm to about 20 mm. The maximum cross-sectional width is used to describe the maximum width at any point of a given cross-section. For example, a maximum cross-sectional width of a trapezoid would be the base width. For example, the flow channel may have a maximum cross-sectional width in mm of about 0.125, 0.25, 0.5, 0.75, 1, 2, 3, 4, or 5, 10, 15, 20 or a value within a range between any of the preceding values, for example, between about 3 and about 4, between about 2 and about 5, or the like. In some embodiments, the flow channel may have a uniform maximum cross-sectional width. In other embodiments, the flow channel may have a non-uniform maximum cross-sectional width. Meaning, a flow channel may or may not increase or decrease in maximum cross-sectional width along the flow channel.

In some embodiments, the flow channel may have a maximum cross-sectional height of about 0.05 mm to about 25 mm. The maximum cross-sectional height is used to describe the maximum height at any point of a given cross-section. For example, a maximum height of a triangle is the distance from peak to base. For example, the flow channel may have a maximum cross-sectional height in mm of about 0.05, 0.1, 0.2, 0.25, 0.5, 0.75, 1, 2, 3, 4, or 5, 10, 15, 20, 25 or a value within a range between any of the preceding values, for example, between about 3 and about 4, between about 2 and about 5, or the like. In some embodiments, the flow channel may have a uniform maximum cross-sectional height. In other embodiments, the flow channel may have a non-uniform maximum cross-sectional height. Meaning, a flow channel may or may not increase or decrease in maximum cross-sectional height along the flow channel.

In some embodiments, the flow channel may have a hydraulic radius of about 0.025 mm to about 12.5 mm. For example, the flow channel may have a hydraulic radius in mm of about 0.025, 0.05, 0.10, 0.20, 0.25, 0.35, 0.45, 0.55, 0.65, 0.75, 0.85, 0.95, 1.05, 1.10, 1.15, 1.20, 1.25, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or 12.5, or a value within a range between any of the preceding values, for example, between about 4.5 and about 11.0, between about 0.35 and about 0.55, or the like. In some embodiments, the flow channel may have a uniform hydraulic radius. In other embodiments, the flow channel may have a non-uniform hydraulic radius. As used herein “non-uniform hydraulic radius” means the flow channel may have a hydraulic radius that fluctuates between the preceding values along the length of the flow channel.

In some embodiments, the flow channel may have any cross-sectional shape. For example, the flow channel may have a cross-sectional shape selected from circle, a semi-circle, a square, a rectangle, a trapezoid, and a triangle. In other embodiments, for example, the flow channel may have a cross-sectional shape designed to increase flow resistance, such as a multi-point star or semi-star. In some embodiments, the flow channel may have a uniform cross-sectional shape. In other embodiments, the flow channel may have a non-uniform cross-sectional shape. Meaning, a flow channel may or may not change in cross-sectional shape along the flow channel.

In some embodiments, the flow channel may be configured to deliver a sterilant to an indicator compartment in any conceivable way. For example, the flow channel may pass over, pass through, or pass into the indicator compartment, or a combination thereof. A flow channel passing over an indicator compartment may have an indicator compartment that interrupts the flow channel such that the sterilant enters the indicator compartment from the top and at least partially fills the indicator compartment before further proceeding along the channel. A flow channel passing through an indicator compartment may have an indicator compartment that interrupts the flow channel such that the sterilant enters the indicator compartment from the side and at least partially fills the indicator compartment before proceeding along the channel. A flow channel passing into an indicator, for example, may have a conduit, e.g., a sub-flow channel, extending from the flow channel with or without having an exit conduit(s) leading away from the indicator compartment. Sterilant may enter an indicator compartment anywhere along the compartment side, top, or bottom.

In some embodiments, the flow channel(s) and/or sub-flow channel(s) may be made from any suitable material that is substantially impermeable to sterilant, e.g., steam, ethylene oxide, vaporized hydrogen peroxide, or the like. For example, the flow channel may include polyethylene terephthalate and a silane-terminated polyurethane adhesive. Other examples of suitable flow channel material may include polyester, cycloolefins, and polycarbonates, or the like.

In some embodiments, the flow channel(s) and/or sub-flow channel(s) may be made from, or include, any suitable material that can at least partly absorb the sterilant, e.g., hydrogen peroxide. A flow channel including a sterilant-absorbing material may allow for an increase in flow resistance. Example hydrogen peroxide-absorbent materials include polyvinylchlorides, polyurethanes, acrylates, engineering SLA (stereolithography) resins, thermoset polymers and the like.

In some embodiments, the flow channel may include one or more sub-flow channel extending from it as described herein.

In some embodiments, for devices having more than one flow channel, each flow channel for any given challenge network may be independently selected from the dimensions and materials described above.

### Sub-Flow Channel

In some embodiments, each sub-flow channel for any given challenge network may be independently selected from the dimensions described below.

In some embodiments, a sub-flow channel may have a length of about 2 mm to about 200 mm. A sub-flow channel length is a measure of the total distance a sterilant may travel. For example, a sub-flow channel may have a length in mm of about 2, 5, 10, 20, 30, 40, 50, 60, 70, 80, 90, 100, 120, 140, 160, or 200, or a value in a range between any of the preceding values, for example, between about 20 and about 60, between about 10 and about 80, or the like.

In some embodiments, a sub-flow channel may have a maximum cross-sectional width of about 0.125 mm to about 20 mm. The maximum cross-sectional width is used to describe the maximum width at any point of a given cross-section. For example, a maximum cross-sectional width of a trapezoid would be the base width. For example, the sub-flow channel may have a maximum cross-sectional width in mm of about 0.125, 0.25, 0.5, 0.75, 1, 2, 3, 4, or 5, 10, 15, 20 or a value within a range between any of the preceding values, for example, between about 3 and about 4, between about 2 and about 5, or the like. In some embodiments, a sub-flow channel may have a uniform maximum cross-sectional width. In other embodiments, the sub-flow channel may have a non-uniform maximum cross-sectional width. Meaning, a sub-flow channel may or may not increase or decrease in maximum cross-sectional width along the sub-flow channel.

In some embodiments, a sub-flow channel may have a maximum cross-sectional height of about 0.05 mm to about 25 mm. The maximum cross-sectional height is used to describe the maximum height at any point of a given cross-section. For example, a maximum height of a triangle is the distance from peak to base. For example, the flow channel may have a maximum cross-sectional height in mm of about 0.05, 0.1, 0.2, 0.25, 0.5, 0.75, 1, 2, 3, 4, or 5, 10, 15, 20, 25 or a value within a range between any of the preceding values, for example, between about 3 and about 4, between about 2 and about 5, or the like. In some embodiments, a sub-flow channel may have a uniform maximum cross-sectional height. In other embodiments, a sub-flow channel may have a non-uniform maximum cross-sectional height. Meaning, a sub-flow channel may or may not increase or decrease in maximum cross-sectional height along the flow channel.

In some embodiments, a sub-flow channel may have a hydraulic radius of about 0.025 mm to about 12.5 mm. For example, a sub-flow channel may have a hydraulic radius in mm of about 0.025, 0.05, 0.10, 0.20, 0.25, 0.35, 0.45, 0.55, 0.65, 0.75, 0.85, 0.95, 1.05, 1.10, 1.15, 1.20, 1.25, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or 12.5, or a value within a range between any of the preceding values, for example, between about 0.45 and about 1.10, between about 0.35 and about 0.55, or the like. In some embodiments, a sub-flow channel may have a uniform hydraulic radius. In other embodiments, a sub-flow channel may have a non-uniform hydraulic radius. As used herein “non-uniform hydraulic radius” means the sub-flow channel may have a hydraulic radius that fluctuates between the preceding values along the length of the sub-flow channel.

In some embodiments, a sub-flow channel may have any cross-sectional shape. For example, the sub-flow channel may have a cross-sectional shape selected from circle, a semi-circle, a square, a rectangle, a trapezoid, and a triangle. In other embodiments, for example, the sub-flow channel may have a cross-sectional shape designed to increase flow resistance, such as a multi-point star or semi-star. In some embodiments, the sub-flow channel may have a uniform cross-sectional shape. In other embodiments, the sub-flow channel may have a non-uniform cross-sectional shape. Meaning, a sub-flow channel may or may not change in cross-sectional shape along the flow channel.

In some embodiments, a sub-flow channel may terminate without passing over, passing through, or passing into one or more indicator compartment.

In some embodiments, a sub-flow channel may terminate at an indicator compartment.

In some embodiments, a sub-flow channel may terminate or pass through at an indicator compartment that does not contain an indicator.

In some embodiments, a sub-flow channel may flow to and from an indicator compartment. In some embodiments, a sub-flow channel flowing from an indicator compartment may flow to another indicator compartment. In some embodiments, a sub-flow channel flowing from an indicator compartment may flow back into the flow channel.

### Indicator Compartments

In some embodiments, a challenge network may include 2-20 indicator compartments, or more. For example, a challenge network may include 2, 3, 4, 5, 6, 7, 8, 9, 10, 11, 12, 13, 14, 15, 16, 17, 18, 19, or 20 indicator compartments, or a value within a range between any of the preceding values, for example, between 5 and 10, between about 8 and 12, or the like.

In embodiments including more than one challenge network, any given challenge network may include 1-20 indicator compartments. For example, a challenge network may include 1, 2, 3, 4, 5, 6, 7, 8, 9, 10, 11, 12, 13, 14, 15, 16, 17, 18, 19, or 20 indicator compartments, or a value within a range between any of the preceding values, for example, between 1 and 5, between about 3 and 6, or the like.

In some embodiments, any indicator compartment within a sterilization monitoring device may have a volume independently selected from about 0.25 cm³ to about 10 cm³. For example, an indicator compartment may have a volume in cm³ of about 0.25, 0.50, 0.75, 1.0, 1.25, 1.50, 1.75, 2.0, 2.25, 2.50, 2.75, 3.0, 3.25, 3.50, 3.75, 4.0, 4.25, 4.50, 4.75, 5.0, 5.25, 5.50, 5.75, 6.0, 6.25, 6.60, 6.75, 7.0, 7.25, 7.50, 7.75, 8.0, 8.25, 8.50, 8.75, 9.0, 9.25, 9.50, 9.75, 10.0, or a value within a range between any of the preceding values, for example, between about 0.5 and about 1.0, between about 1.0 and about 1.0 and about 2.0, or the like.

In some embodiments, the indicator compartment(s) may be arranged in any conceivable way within a challenge network.

In some embodiments, a challenge network may include one or more empty indicator compartments. An empty indicator compartment may simply serve to increase the cavity volume, thereby increasing the flow resistance of the challenge network.

In some embodiments, the indicator compartments may include a pointed end configured to pierce a barrier (e.g., foil) separating the indicator compartment from a medium compartment including a medium described herein, e.g., nutrient medium. In such embodiments, a user may activate the device by way of, for example, applying pressure or maneuvering a switch, such that the pointed end pierces the barrier and allows for the indicator to interact with the medium.

In some embodiments, the indicator compartments may be made from any material that is substantially impermeable to the sterilant. For example, the indicator compartments may include a polymeric composition. For example, the polymeric composition may be or include polypropylene, polyester, cycloolefins, polycarbonates, or the like.

### Indicators

In some embodiments, each indicator compartment may independently include an indicator selected from a steam indicator, an ethylene oxide indicator, a hydrogen peroxide indicator, and a combination thereof. In other embodiments, any available indicator may be used for any known sterilant.

In some embodiments, the indicators may be selected from chemical indicators, biological indicators, or a combination thereof.

In some embodiments, the sterilization monitoring device may include at least one biological indicator described herein.

In some embodiments, the sterilization monitoring device may include at least one chemical indicator described herein.

In some embodiments, the sterilization monitoring device may include at least one biological indicator described herein and at least one chemical indicator described herein.

In some embodiments, one or more indicator compartment may include a steam indicator. Various steam indicators are known in the art. Any of such known steam indicators may be used in the sterilization monitoring devices described herein. In some embodiments, the steam indicator may include Bacillus stearothermophilus, Geobacillus stearothermophilus spores, Bacillus atrophaeus spores, a combination thereof, or the like. In some embodiments, the steam indicator may include 2-ethoxybenzamide or salicylamide. In some interactions between a steam indicator and sterilant involves production of sulfur anions that react with lead or another metal to make a black metal sulfide. For example, lead thiosulfate (white) yields lead sulfide (black), copper thiosulfate (yellow) yields copper sulfide (black), ferrous thiosulfate (green) yields ferrous sulfide (black), nickel thiosulfate (green) yields nickel sulfide (black/green), cobalt thiosulfate (red/purple) yields cobalt sulfide (purple/black), bismuth thiosulfate (orange/brown) yields bismuth sulfide (black), chromium thiosulfate (grey/blue) yields chromium sulfide (green), and silver thiosulfate (brown) yields silver sulfide (black) under steam sterilization conditions.

In some embodiments, one or more indicator compartment may include an ethylene oxide indicator. Various ethylene oxide indicators are known in the art. Any of such known ethylene oxide indicators may be used in the sterilization monitoring devices described herein. A typical interaction between an ethylene oxide indicator and sterilant involves reaction of a dye or pigment with ethylene oxide, which results in a color change. Bacillus subtilis var. niger is a commonly-used organism for monitoring ethylene oxide sterilization protocols. 4(4-nitrobenzyl)pyridine and other pyridines and quinolines have been used (see Brewer, et. Al., J. Pharmaceutical Sciences 1966, p. 57-59; U.S. Pat. No. 3,627,469, each incorporated herein by reference in their entireties). Magnesium chloride (MgCl₂)-based inks, and hydrates thereof, have been reported to undergo oxidation to magnesium hydroxide (Mg(OH)₂) (see U.S. Pat. No., 3,098,751 incorporated herein by reference in its entirety). Other examples of ethylene oxide indicators include iron chloride (FeCl₂) and zinc chloride (ZnCl₂), or hydrates thereof. Another ethylene oxide indicator is Bacillus atropheus.

In some embodiments, one or more indicator compartment may include a hydrogen peroxide indicator, i.e., an indicator for vaporized hydrogen peroxide. Various hydrogen peroxide indicators are known in the art, such as those listed in paragraph [00123] below, e.g., Geobacillus stearothermophilus. Any of such known hydrogen peroxide indicators may be used in the sterilization monitoring devices described herein. A typical interaction between a hydrogen peroxide indicator and sterilant involves oxidation of the indicator dye or pigment. Another approach involves oxidation of a metal salt to make highly reactive oxygen radicals that subsequently react with the dye or pigment. The chemical-indicating composition of the present disclosure may contain more than one dye or pigment, or a mixture of one or more dye and one or more pigment. An example of a mixture of a dye and a pigment is an indicating composition that contains a pigment that is stable to interaction with hydrogen peroxide and a dye that reacts with hydrogen peroxide. For example, a red pigment can be combined with an excess amount of blue dye to make a predominately blue chemical-indicating composition, which upon interaction with hydrogen peroxide turns pink due to bleaching of the blue dye (to colorless) by the action of hydrogen peroxide, thus revealing the red pigment. In some embodiments, the dye or pigment may be chosen from methane, monoazo, diazo, triazo, diazine, thiazine, cyanine, xanthene, oxazine, anthraquinone, benzodifuranone, phthalocyanine, quinophthalone, and nitro- and nitroso colorants, Victoria green S extra, Basic blue 41, Basic red 15, Acid green AX986, Keystone soap fluoro green, Basic red 14, copper salts, cobalt salts, iron salts, chromium salts, Patent blue violet, Alkali blue 4B, Victoria pure blue BO, Acid fuchsin sodium salt, Alphaszurine A, Methyl violet 2B, Ethyl violet, FD/C blue 1, Brilliant blue R, Lissamine green B, Erioglaucine, Eriochrome black T, Eriochrome blue black B, Cibacron brilliant red 3B, Chromotrope 2B, Amaranth, D&C red No. 33, Bordeaux R, Acid violet 7, Acid violet 5, Plasmocorinth B, Acid Blue 113, Acid red 151, Acid black 24, Acid red 97, Direct red 75, Brilliant crocein MOO, Ponceau SS, Reactive black 5, Arsenazo 111, Direct blue 71, Azocarmine G, Methylene violet 3RAX, Toluidine blue O, Methylene green, Sulforhodamine B, Rhoadanine 6G, Violamine R, Nile blue A, Basic blue 3, Brilliant cresyl blue BB, Alizarin violet 3R, D7C green No. 5, and a combination thereof.

In some embodiments, one or more indicator compartment may include a biological indicator. Any biological indicators known in the art may be used in the sterilization monitoring devices described herein. Example biological indicators include Geobacillus stearothermophilus spores, Bacillus atrophaeus spores, Aspergillus niger, Bacillus subtilis, Clostridium spp., Bacillus subtilis var. niger, Bacillus stearothermophilus, or a combination thereof, or the like.

In some embodiments, one or more indicator compartment may include a chemical indicator. Any chemical indicators known in the art may be used in the sterilization monitoring device described herein. Example chemical indicators include those described above. In some embodiments, a wicking strip may be provided near the chemical indicator such that upon melting, the chemical may travel up the wick at a rate proportional to the sterilization temperature and time of exposure.

In some embodiments, a portion of indicator compartments may include a biological indicator, and a portion of indicator compartments may include a chemical indicator.

In some embodiments, the indicators may be present at any concentration or population so as to allow for tailoring of the sterilization monitoring device to a particular protocol.

### Medium Compartments, Ports, and Packages

In some embodiments, a sterilization monitoring device may further include one or more medium compartment having a medium therein. The one or more medium compartment may be in fluid communication with one or more indicator compartment. In some embodiments, the medium is selected from a nutrient medium, a fluorogenic medium, a pH indicator medium, or a combination thereof.

In some embodiments, a medium compartment may be separated from the indicator compartment(s) by a removable barrier. The removable barrier may be in the form of a layer that can be pierced, e.g., a foil layer, or otherwise punctured with pressure. In other embodiments, a removable barrier may be in the form of a retractable barrier.

In some embodiments, a medium compartment may be situated above or below an indicator compartment. In other embodiments, a medium compartment may be in fluid connection with one or more indicators by way of a medium pathway.

In some embodiments, a medium compartment may be situated within the flow channel path such that sterilant passes through the medium compartment thereby contributing to the overall cavity volume of the sterilization monitoring device.

In embodiments where the sterilization monitoring device includes a medium port for receiving a medium, the medium port may be of any configuration suitable for receiving a medium. For example, the medium port may be a mechanical valve. For example, the medium port may be a puncturable valve, such as those under the tradename Sure/Seal™ commonly used on Aldrich® bottles containing air and/or moisture-sensitive reagents. In such embodiments, a medium package may be used to deliver the medium to the sterilization monitoring device through the medium port. The medium package may be a container (and means to transfer medium from container to medium port), a foil pouch, a pre-loaded syringe or pipette, or the like. In some embodiments, the medium package may include a medium output for direct dispensing of the medium from the medium package to the medium port, e.g., a needle or spout configured to mate with the medium port.

### Mediums

In some embodiments, the medium may include any medium suitable for promoting a detectable change in one or more indicator when the indicator is in the presence of the medium.

In some embodiments, the medium may include a nutrient medium. In some embodiments, the nutrient medium may be suitable for promoting the growth of microorganisms. After the sterilization process is a complete, a nutrient medium may be added to a biological indicator and incubated under conditions effective to promote growth of any surviving test microorganisms. If the sterilization cycle failed, surviving microorganisms would be available to undergo further biological transformations in order to generate a detectable signal. Signal detection would indicate that the sterilization cycle was not effective.

In some embodiments, the nutrient medium may be any nutrient medium known in the art. A nutrient medium may include any nutrients known to promote spore germination and proliferation. The nutrient medium may include one or more sugars, e.g., glucose, fructose, cellobiose, or the like. The nutrient medium may further include salts, such as potassium chloride, calcium chloride, or the like. The nutrient medium may further include at least one amino acid, for example, methionine, phenylalanine, tryptophan, or the like. Growth media components and concentrations are known and described, for example, in WO 99/05310 (Tautvydas) and Zechman et al., J. Food, Sci., 56, 5, p. 1408-1411 (1991).

In some embodiments, the medium may include a fluorogenic compound. In the presence of a fluorogenic compound, surviving microorganisms may interact with the fluorogenic compound to produce a detectable fluorescence signal. A fluorogenic medium is a medium having one or more compounds suitable for use in identifying enzymatic activity by producing fluorescence in the presence of such enzymatic activity. Enzymatic activity is indicative of the presence of spores. In some embodiments, the fluorogenic medium may include 4-methylumbelliferyl-2-acetamido-4, 6-O-benzylidene-2-deoxy-.beta.-D-glucopyranoside; 4-methylumbelliferyl acetate; 4-methylumbelliferyl-N-acetyl-.beta.-D-galactosaminide; 4-methylumbelliferyl-N-acetyl-.alpha.-D-glucosaminide; 4-methylumbelliferyl-N-acetyl-.beta.-D-glucosaminide; 2′-(4-methylumbelliferyl)-.alpha.-D-N-acetyl neuraminic acid; 4-methylumbelliferyl-.alpha.-L-arabinofuranoside; 4-methylumbelliferyl-.alpha.-L-arabinoside; 4-methylumbelliferyl butyrate; 4-methylumbelliferyl.beta.-D-cellobioside; methylumbelliferyl-.beta.-D-N,N′-diacetyl chitobioside; 4-methylumbelliferyl elaidate; 4-methylumbelliferyl-.beta.-D-fucoside; 4-methylumbelliferyl .alpha.-L-fucoside; 4-methylumbelliferyl-.beta.-L-fucoside; 4-methylumbelliferyl-.alpha.-D-galactoside; 4-methylumbelliferyl-.beta.-D-galactoside; 4-methylumbelliferyl-.alpha.-D-glucoside; 4-methylumbelliferyl-.alpha.-D-glucoside; 4-methylumbelliferyl-.beta.-D-glucuronide; 4-methylumbelliferyl-p-guanidinobenzoate; 4-methylumbelliferyl heptanoate; 4-methylumbelliferyl-.alpha.-D-mannopyranoside; 4-methylumbelliferyl-.beta.-D-mannopyranoside; 4-methylumbelliferyl oleate; 4-methylumbelliferyl palmitate; 4-methylumbelliferyl phosphate; 4-methylumbelliferyl propionate; 4-methylumbelliferyl stearate; 4-methylumbelliferyl sulfate; 4-methylumbelliferyl-.beta.-D-N, N′, N″-triacetylchitotriose; 4′-methylumbelliferyl-2,3,5 -tri-o-benzoyl-.alpha.-L-arabinofuranoside; 4-methylumbelliferyl-p-trimethylammonium cinnamate chloride; and 4-methylumbelliferyl-.beta.-D-xyloside, or a combination thereof.

In some embodiments the fluorogenic medium may include L-alanine-7-amido-4-methylcoumarin; L-proline-7-amido-4-methylcoumarin; L-tyrosine-7-amido-4-methylcoumarin; L-leucine-7-amido-4-methylcoumarin; L-phenylalanine-7-amido-4-methylcoumarin; 7-glutaryl-phenylalanine-7-amido-4-methylcoumarin; N-t-BOC-Ile-Glu-Gly-Arg-7-amido-4-methylcoumarin; N-t-BOC-Leu-Ser-Thr-Arg-7-amido-4-methylcoumarin; N-CBZ-Phe-Arg-7-amido-4-methylcoumarin; Pro-Phe-Arg-7-amido-4-methylcoumarin; N-t-BOC-Val-Pro-Arg 7-amido-4-methylcoumarin; and N-glutaryl-Gly-Arg 7-amido-4-methylcoumarin.

In some embodiments, the fluorogenic medium may include fluorescein diacetate, fluorescein di-(B-D-galacto-pyranoside), fluorescein dilaurate, or a combination thereof.

In some embodiments, the medium may further include a pH indicator. In the presence of a pH indicator, surviving microorganisms may interact with the pH indicator to produce a detectable color change. A pH indicator medium includes a compound(s) demonstrating different colors depending on the pH of the solution. An increase in acid in the presence of metabolically active spores can easily be visualized with a pH indicator medium. In some embodiments, the pH indicator medium may be selected from cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-tolylazo-o-toluidine, quinaldine red, 2,4-dinitrophenol, erythrosine disodium salt, benzopurpurine 4B, N,N-dimethyl-p-(m-tolylazo) aniline, p-dimethylzminoazobenzene, 4,4′-bis(2-amino-1-naphthylazo)-2,2′-stilbenedisulfonic acid, tetrabromophenolphthalein ethyl ester potassium salt, bromophenol blue, congo red, methyl orange, ethyl orange, 4-(4-dimethylamino-1-naphylazo-3-methoxybenzenesulfonic acid, bromocresol green, resazurin, 4-phenylazo-1-naphthylamine, ethyl red, 2-(p-dimethylaminophenylazo) pyridine, 4-(p-ethoxyphenylazo)-m-phenylene-diamine monohydrochloride, resorcin blue, alizarin red S, methyl red, propyl red, bromocresol purple, chlorophenol red, p-nitropheno, alizarin, 2-(2,4-dinitrophenylazo)-1-naphthol-3,6-disulfonic acid disodium salt, bromothymol blue, 6,8-dinitro-2,4-(1H)-quinazolinedione, brilliant yellow, phenol red, neutral red, m-nitrophenol, cresol red, turmeric, metacresol purple, 4,4′-bis(4-amino-1-naphthylazo)-2,2′-stilbenedisulfonic acid, thymol blue, p-naphthobenzein, phenolphthalein, o-cresolphthalein, ethyl bis-(2,4-dimethylphenyl) ethanoate, thymolphthalein, alizarin yellow R, alizarin, p-(2,4-dihydroxyphenylazo) benzenesulfonic acid sodium salt, 5,5′-indigodisulfonic acid disodium salt, 2,4,6-trinitrotoluene, 1,3,5-trinitrobenzene, clayton yellow, or the like, and a combination thereof.

In some embodiments, a medium compartment may include a nutrient medium and a fluorogenic medium.

In some embodiment, a medium compartment may include a nutrient medium and a pH indicator medium.

In some embodiments, a medium compartment may include a nutrient medium, a fluorogenic medium, and a pH indicator medium.

### Shell

In some embodiments, the shell may be made from any material that is substantially impermeable to the sterilant, such as the sterilant-impermeable materials described above. For example, the shell may include a polymeric composition. For example, the polymeric composition may be or include polypropylene.

In some embodiments, the shell may include a removable lid. A removeable lid may be, for example, a polymeric film secured to a shell base with an adhesive, wherein the adhesive is substantially impermeable to sterilant. Alternatively, a removable lid may be joined to a shell base by a gasket, wherein the gasket is substantially impermeable to sterilant. A removable lid may allow a user to open the sterilization monitoring device in order to add or remove components for further evaluation.

In other embodiments, the shell may include a non-removeable lid. A non-removeable lid may be, for example, thermosealed to a shell base.

In some embodiments, the shell may include a transparent lid that is removeable or non-removeable.

### Evaluation Methods

In many embodiments, a method for evaluating a sterilization process is described. The method may include providing a sterilization monitoring device described herein. The method may include exposing the sterilization monitoring device to conditions set forth in a sterilization protocol. The sterilization protocol conditions may include: allowing a sterilant described herein to contact the sterilization monitoring device for a period; inspecting one or more indicators for a change in one or more of color, pH, and fluorescence; comparing the change in one or more of color, pH, and fluorescence with a sterilization threshold value; and determining if the change in one or more of color, pH, and fluorescence indicates a satisfactory sterilization in view of the sterilization threshold value.

In some embodiments, the method may further include contacting one or more indicator compartment with a nutrient medium described herein, a fluorogenic medium described herein, a pH indicator medium described herein, or a combination thereof prior to the inspecting.

In some embodiments, the method may further include activating the sterilization monitoring device to allow for the contacting of the one or more indicator compartment with medium(s) described herein. For example, the method may further include removing a lid from the sterilization monitoring device prior to contacting one or more indicator compartment with the medium(s). For example, the method may further include removing a barrier separating a medium compartment from one or more indicator compartment prior to contacting one or more indicator compartment with the medium(s). Removing a barrier may include, for example, puncturing a foil seal separating an indicator compartment and a medium compartment. Further, for example, a medium compartment external to the sterilization monitoring device may be inserted and the contents expelled into the sterilization monitoring device.

In some embodiments, the method may further include incubating one or more indicator with one or more of a nutrient medium described herein, a fluorogenic medium described herein, and a pH indicator medium described herein at a temperature for a period. In some embodiments, the temperature may be from about 40° C. to about 100° C., e.g., 40, 45, 50, 55, 60, 65, 70, 75, 80, 85, 90, 95, or 100, or a value between any of the preceding values. In some embodiments, the period may be from about 15 min to about 90 min, e.g., 15, 20, 25, 30, 35, 40, 45, 50, 55, 60, 65, 70, 75, 80, 85, or 90 min, or a value between any of the preceding values.

In some embodiments, the method may further include inspecting one or more indicators for a change in color, pH, and fluorescence after incubating one or more indicator with one or more of a nutrient medium, a fluorogenic medium, and a pH indicator medium at a temperature for a period.

In some embodiments, the method may further include inspecting one or more indicators for a change in fluorescence after contacting one or more indicator with one or more of a nutrient medium, a fluorogenic medium, and a pH indicator medium prior to incubating, and further inspecting one or more indicators for a change in color and pH after incubating.

In some embodiments, the conditions set forth in the sterilization protocol may include contacting the sterilization monitoring device with one or more of steam, ethylene oxide gas, and hydrogen peroxide gas at a temperature and pressure for a period. The contacting may under any sterilant conditions described herein.

In some embodiments, the sterilization threshold value may refer to a number of indicators required to be changed as a factor of one or more of 1) cavity volume; 2) hydraulic radius; 3) flow resistance; 4) load size; 5) load contaminants; 6) load having challenging objects with respect to shape; 7) temperature; and 8) pressure.

In some embodiments, the sterilization protocol may be for sterilizing hospital equipment, e.g., utensils and/or supplies. For example, the hospital equipment may include linens, e.g., towels, bed sheets, blankets, pillows, garments, footwear, coats, gloves, or the like. For example, the hospital equipment may include surgical instruments, e.g., clamps, scissors, knives, forceps, blades, or the like. For example, the hospital equipment may include any equipment that may come in contact with a patient or their surroundings, e.g., stethoscopes, bed pans, face shields, goggles, dinnerware, or the like. For example, the hospital equipment may include irregular-shaped equipment or equipment having an inner cavity, e.g., needles, endoscopes, tubing, cannulas, or the like.

In some embodiments, the sterilization protocol may be for sterilizing laboratory equipment. For example, the laboratory equipment may include laboratory glass-, ceramic- or plastic-ware, e.g., beakers, flasks, funnels, pipettes, burets, petri dishes, collection containers, test tubes, chromatography columns, or the like. For example, the laboratory equipment may include laboratory metalware, e.g., spatulas, forceps, stirbars, needles, clamps, ringstands, tubing, or the like.

### Sterilant

In many embodiments, any sterilization monitoring device may be configured to accept any sterilant known under any sterilization protocol available.

In some embodiments, the sterilant may be selected from steam, ethylene oxide, and hydrogen peroxide.

In some embodiments, the sterilant may be in gaseous form.

In some embodiments, the sterilant may be steam at a temperature of about between about 120° C. to about 140° C. In some embodiments, the pressure may be below about 0.5 bar. In some embodiments, the period may be from about 10 to about 60 min. In other embodiments, the period may be from about 3 to about 40 min.

In some embodiments, the sterilant may be gaseous ethylene oxide at a temperature of about 35° C. to about 65° C. In some embodiments the ethylene oxide concentrations may be from about 450 mg/L to about 1,200 mg/L. In some embodiments, the sterilant may further include steam, i.e., water in about 40 to about 80%). In some embodiments, the period may be from about 1 to about 6 hr.

In some embodiments, the sterilant may be vaporized hydrogen peroxide at a temperature of about 6° C. to about 60° C. In some embodiments, the pressure may be between about 1000 mbar and about 0.3 mbar.

In other embodiments, the sterilant may be in liquid form.

### Kits

In many embodiments, a kit is described. The kit may include a sterilization monitoring device described herein. The kit may further include instructions directing a user to perform the steps of a method for evaluating the efficacy of a sterilization protocol described herein.

In some embodiments, the kit may further include a nutrient medium described herein. In some embodiments, the nutrient medium may be included in a separate container. In other embodiments, the nutrient medium may be included within the sterilization monitoring device. The grow medium may be any nutrient medium described herein.

In some embodiments, the kit may further include a fluorogenic medium. In some embodiments, the fluorogenic medium may be included in a separate container. In other embodiments, the fluorogenic medium may be included within the sterilization monitoring device. The fluorogenic medium may include any fluorogenic compound described herein.

In some embodiments, the kit may further include a pH indicator medium. In some embodiments, the pH indicator medium may be included in a separate container. In other embodiments, the pH indicator medium may be included within the sterilization monitoring device. The pH indicator medium may include any pH indicator described herein.

In some embodiments, the kit may further include one or more medium package described herein. The medium package(s) may include a nutrient medium, a fluorogenic medium, a pH indicator medium, or a combination thereof.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

### Example 1

A sterilization monitoring device having 8 indicator compartments, similar to the device illustrated in FIG. 1A-C, was prepared out of polypropylene well strips (indicator compartments) with laminated layers of film to create a flow channel. Spores were coated within each of the indicator compartments (volume 0.36 cm³) prior to applying the laminate film. Geobacillus stearothermophilus spores (500 μL) were centrifuged at 7000 rpm for 2 min and the supernatant was removed. The spores were re-suspended in a coating solution consisting of trehalose and phosphate buffered saline. The coating solution (3 μL) was spotted onto the bottom of each indicator compartment (8-well strip, polypropylene KingFisher, Cat. #97002084) and dried at 54° C. for 15 min. Polyethylene terephthalate film (0.254 mm×2) with silane-terminated polyurethane (SPU) adhesive was laser cut to form 5 mm wide×0.254 deep flow channel. The flow channel was adhered to the top of the indicator compartments such that the flow channel is the only path for a sterilant to contact the spores.

Four of such sterilization monitoring devices were exposed to ISO 132° C. pre-vac cycles (steam) in an H&W 105 resistometer for 1 min, 2 min, 3 min, and 4 min, respectively. The devices were opened, and a nutrient medium including pH indicator was added to each indicator compartment. The devices were incubated at 60° C. overnight in a sealed bag with a moistened paper towel. The spores in each indicator compartment in the devices exposed to 2-4 min of steam were killed, whereas the two compartments opposite the flow channel entry were not killed after exposure to 1 min of steam.

### Example 2

The sterilization monitoring device described in Example 1 was prepared with the exception that the flow channel was prepared with different dimensions, i.e., 0.508 mm wide×0.254 mm deep. Geobacillus stearothermophilus spores (3M Brookings, SD) were centrifuged at 6000 rpm for 2 min and the supernatant was removed. The spores were re-suspended in consisting of consisting of trehalose and phosphate buffered saline. The coating solution (3 μL) was spotted onto the bottom of each indicator compartment and dried at 56° C. for 20 min.

Three of such sterilization monitoring devices were exposed to steam in a Midmark M9 121° C. pre-vac cycles for 0 min, 3 min, and 6 min, respectively. Upon completion of the sterilization cycles, the device was opened and nutrient medium containing 4-methylumbelliferyl glucuronide (4-MUG; fluorogenic compound) was added. Fluorescence data is provided in FIGS. 7 and 8 for the 3 min and 6 min cycle, respectfully (steam entered through indicator compartment H and moved toward indicator compartment A). The plate was placed in a 60° C. incubator overnight for growth measurement. At zero minutes of steam exposure, spores in each indicator compartment survived. At three minutes of steam exposure, spores in all but the first indicator compartment survived. At six minutes of steam exposure, spores in all but the first two indicator compartment survived. It is believed that the decrease in spore kill may be due to the increase in flow resistance of the smaller flow channel. This shows that devices may be tailored and calibrated to be suitable for a variety of sterilization loads.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A sterilization monitoring device comprising:
a challenge network comprising:
a flow channel comprising an entry port for receiving a sterilant, and
a plurality of indicator compartments,
each indicator compartment comprising one or more indicator, and
each indicator compartment being in fluid communication with the flow channel; and
a shell encasing the challenge network such that access to the plurality of indicator compartments is only through the flow channel.

2. A sterilization monitoring device comprising:
a plurality of challenge networks, each challenge network comprising:
a flow channel comprising an entry port for receiving a sterilant, and
one or more indicator compartment comprising one or more indicator,
wherein at least one indicator is a biological indicator;
at least one medium compartment comprising a nutrient medium,
wherein the medium compartment is in fluid communication with one or more indicator compartment; and
a shell encasing the challenge network such that access to the one or more indicator compartment is only through the flow channels.

3. (canceled)

4. (canceled)

5. (canceled)

6. (canceled)

7. (canceled)

8. The sterilization indicator device of claim 1, the flow channel characterized by one or more:
a length of about 10 mm to about 800 mm,
a maximum cross-sectional width of about 0.125 mm to about 20 mm, wherein the width is uniform or non-uniform along a length of the flow channel, and
a maximum cross-sectional height of about 0.05 mm to about 25 mm, wherein the height is uniform or non-uniform along a length of the flow channel.

9. (canceled)

10. The sterilization indicator device of claim 1, the flow channel having a hydraulic radius of about 0.025 mm to about 12.5 mm, wherein the hydraulic radius is uniform or non-uniform along a length of the flow channel.

11. (canceled)

12. (canceled)

13. The sterilization monitoring device of claim 1, wherein the flow channel is comprised of a polyethylene terephthalate film and a silane-terminated polyurethane adhesive.

14. The sterilization monitoring device of claim 1, further comprising one or more sub-flow channel extending from the flow channel.

15. (canceled)

16. (canceled)

17. The sterilization monitoring device of claim 14, each sub-flow channel independently characterized by one or more:
a length of about 2 mm to about 200 mm,
a maximum width at any point of about 0.125 mm to about 20 mm, wherein the width is uniform or non-uniform along the length of the sub-flow channel, and
a height at any point of about 0.05 mm to about 25 mm, wherein the height is uniform or non-uniform along the length of the sub-flow channel.

18. (canceled)

19. The sterilization monitoring device of claim 1, each indicator compartment independently having a volume between about 0.25 cm³ and about 10 cm³.

20. The sterilization monitoring device of claim 1, one or more indicator compartments independently comprising an indicator selected from a steam indicator, an ethylene oxide indicator, and a hydrogen peroxide indicator, the indicator being a biological indicator, a chemical indicator, or a combination thereof.

21. (canceled)

22. The sterilization monitoring device of any one of claim 1, wherein one or more indicator compartment comprises a biological indicator selected from Geobacillus stearothermophilus spores, Bacillus atrophaeus spores, Aspergillus niger, Bacillus subtilis, Clostridium spp., Bacillus subtilis var. niger, and a combination thereof.

23. (canceled)

24. The sterilization monitoring device of claim 1, wherein one or more indicator compartment independently comprises a chemical indicator selected from a coloring-changing dye, a color-changing pigment, a metal sulfide precursor(s), a metal complex or salt, a fluorescent molecular switch, and a combination thereof.

25. The sterilization monitoring device of claim 1, further comprising at least one medium compartment comprising a medium selected from a nutrient medium, a fluorogenic medium, a pH indicator medium, and a combination thereof, wherein the medium compartment is in fluid communication with one or more indicator compartment.

26. The sterilization monitoring device of claim 1, further comprising:
a medium port for receiving a medium selected from a nutrient medium, a fluorogenic medium, a pH indicator medium, and a combination thereof, and
a medium path, wherein one or more indicator compartment is in fluid communication with the medium port via the medium path.

27. (canceled)

28. The sterilization monitoring device of claim 25, further comprising at least one removeable barrier separating the at least one medium compartment from the one or more indicator compartment.

29. (canceled)

30. (canceled)

31. (canceled)

32. The sterilization monitoring device of claim 1, further comprising a package that is at least partly permeable to the sterilant, wherein the package at least partly surrounds the shell.

33. A method for evaluating a sterilization process, the method comprising:
providing a sterilization monitoring device of claim 1;
exposing the sterilization monitoring device to conditions set forth in a sterilization protocol;
allowing a sterilant to contact the sterilization monitoring device at a temperature for a period;
inspecting one or more indicators for a change in one or more of color, pH, and fluorescence;
comparing the change in one or more of color, pH, and fluorescence with a sterilization threshold value; and
determining whether or not the change in one or more of color, pH, and fluorescence indicates a satisfactory sterilization in view of the sterilization threshold value.

34. The method of claim 33, further comprising contacting one or more indicators with a nutrient medium, a fluorogenic medium, a pH indicator medium, or a combination thereof prior to the inspecting.

35. (canceled)

36. (canceled)

37. (canceled)

38. (canceled)

39. (canceled)

40. (canceled)

41. A kit comprising:
a sterilization monitoring device of claim 1; and
instructions directing a user to: expose the sterilization monitoring device to conditions set forth in a sterilization protocol;
allow a sterilant to contact the sterilization monitoring device at a temperature for a period;
inspect one or more indicators for a change in one or more of color, pH, and fluorescence;
compare the change in one or more of color, pH, and fluorescence with a sterilization threshold value; and
determine whether or not the change in one or more of color, pH, and fluorescence indicates a satisfactory sterilization in view of the sterilization threshold value.

42. The kit of claim 41, further comprising a container comprising nutrient medium, a fluorogenic medium, or a pH indicator medium.

43. (canceled)

44. (canceled)

45. The kit of claim 41, further comprising a medium packet comprising a medium selected from a nutrient medium, a fluorogenic medium, a pH indicator, and a combination thereof, wherein the medium packet is configured to mate with the sterilization monitoring device in order to dispense the medium into the sterilization monitoring device.
